(19) United States
(12) Patent Application Publication
(10) Pub. No.: US 2006/0182809 A1
(43) Pub. Date: Aug. 17, 2006

(54) System for transporting active substances in a biological system

(75) Inventors: Helmut Blum, Dusseldorf (DE); Marcel Roth, Dusseldorf (DE); Wolfgang Greb, Dusseldorf (DE)

Correspondence Address: GUDRUN E. HUCKETT DRAUDT, LONSSTR. 53, WUPPERTAL, 42289, DE

(73) Assignee: EUCRO EUROPEAN CONTRACT RESEARCH GMBH, Neuss (DE)

(21) Appl. No.: 11/308,484

(22) Filed: Mar. 29, 2006

Related U.S. Application Data
(63) Continuation of application No. 10/240,484, filed on Apr. 21, 2003.

(30) Foreign Application Priority Data
Apr. 3, 2000 (DE) 10016559.1

(51) Int. Cl. A61K 9/14; A61K 38/00; A61K 38/43; A61K 39/395

(52) U.S. Cl. 424/489; 424/130.1; 514/2; 424/94.1

(57) ABSTRACT

A stabilizer-free system for transporting active substances in a biological system of one or more active substances and magnetic particles is characterized in that the particles are provided with active substance(s) on at least part of their surface. Modification of the magnetic particles is not absolutely required in this system. It can be incorporated both into aqueous and oily suspensions and into microemulsions, oil-in-water emulsions, water-in-oil emulsions and also water-in-oil-in-water emulsions.

This application is a continuation of U.S. application Ser. No. 10/240,484 having a filing date of Apr. 21, 2003, which U.S. application is a national stage filing of international application PCT/EP01/03318 having an international filing date of Mar. 23, 2001. The instant application incorporates by reference the entire disclosure of the parent application Ser. No. 10/240,484.

### BACKGROUND OF THE INVENTION

The present invention relates to a system for transporting active substances in a biological system, comprising an active substance and magnetic particle(s), to a method for preparing this system and to the use of the system for the transport of pharmaceutical active ingredients and active substances.

Ferrofluids mean magnetic fluids in which a ferr- or ferromagnetic substance, normally magnetite, is dispersed as a colloidally dispersed phase in a liquid dispersion medium such as water, paraffin, toluene or any other liquid including mercury. Frequently, a surfactant or wetting agent such as oleic acid is added to such a mixture in order to prevent agglomeration and thus the formation of larger aggregates. Owing to the small size of the crystallites, the particles behave in a superparamagnetic fashion, i.e. permanent magnetization is not possible.

For many years, the ferrofluids and also other metallic colloids have been the subject of intensive research, since they are capable of binding macromolecules, in particular proteins.

Another field of use for the ferrofluids is the removal of undesired cells, in particular cancer cells, from cell suspensions. In the case of cancer treatment, for example, it is possible to avoid a multiplicity of problems such as the use of toxins, chemotherapeutic agents, etc. by removing cells, with methods of this kind being limited to physical treatment. Density gradient separation has been used for removing lymphocytes from bone marrow cells, but without great efficiency. Various types of magnetic particles are also used in immunoassays, in drug targeting and for removing cells. The most frequently used magnetic material is magnetite which is incorporated into a multiplicity of support systems or microspheres or is bound directly to antibodies. Usually, the magnetic particles are embedded in appropriate coating substances such as polymers or silica gels.

The European Patent Application EP 0 156 537 discloses magnetic colloidal fluids in which the magnetic phase is colloidally dispersed in a liquid dispersion medium. The magnetic phase comprises fine magnetic particles which are coated with a crosslinked, biologically compatible polymer.

The German Patent Application DE 43 07 262 describes a method for preparing magnetic polymeric silicon dioxide in which magnetic materials, in particular Fe, β-Fe₂O₃, γ-Fe₂O₃, Fe₃O₄, are dispersed in alkali metal silicate solutions or are added as a colloidal solution and are precipitated and condensed with mineral acids or carbon dioxide. It is possible to apply X-ray contrast agents to the surface of the particles obtained. They are employed in ultrasound and NMR diagnostic imaging methods and also for concentrating radioactive isotopes. Another field of use is the extracorporeal or intracorporeal export or binding of biological components, toxic substances, bacteria or viruses from the organism with the aid of magnetic fields.

The German Patent Application DE 43 25 386 discloses a magnetic fluid (ferrofluid) on the basis of an aqueous support fluid, in which magnetic iron oxide particles which are mainly composed of magnetite are stabilized by a first monomolecular adsorptive layer of saturated or unsaturated fatty acids and a second adsorptive layer of surface-active substances. Both the first and the second adsorptive layers consist of surfactants which are prepared entirely from naturally sustainable raw materials. The described aqueous magnetic fluid can be used in medicine as a marker substance and/or for transporting active ingredients.

An object of the particles described in the prior art is the targeted transport of substances such as pharmaceutical active ingredients in the organism and the concentration thereof at sites of interest. The said transport can be accomplished essentially by means of two methods, namely on the one hand via concentration by means of antibodies immobilized on the particulate surface or by means of a permanent magnetic field.

The known magnetic particles used are normally polymer particles with sizes of above 500 nm, which are composed of an inorganic or organic polymer and incorporated magnetic particles. Usually, smaller particles are not employed, since particles of a particle size of below 100 nm can, if stabilized, barely be concentrated by means of a magnetic field.

An important field of use of the magnetic or superpara-magnetic particles in the field of medicine is chemotherapy for cancer treatment or the protection of implants by means of antibiotics. The active ingredients are attached by swelling of the polymer particles or by adsorption to the polymeric surfaces. It is also possible to use the magnetic particles suspended in aqueous medium, in which case surface-active substances which keep the magnetic particles and, where appropriate, active ingredients adsorbed thereto in suspension are usually attached to the particle surface.

As described previously, the magnetic particles known in the prior art, which are used for concentration or targeted transport of substances in biological systems, have a modified surface. The modification of the surface serves to keep the said substances in suspension and to bind the active substances. The modification of the surfaces has the disadvantage of this modification being an additional step during preparation. Furthermore, there is the risk of the polymers used for modifying the surfaces being able to interact with the active substances and possibly also with proteins etc. present in the biological system, i.e. in the organism, which interaction impairs the efficacy of the active substances and, in some cases, may even result in undesired, possibly toxic secondary reactions and actions of these agents.

A further disadvantage is the fact that the subsequent application of a coating to already surface-modified particles is complicated and therefore time-consuming. Finally, the coated particles have a relatively low adsorptive capacity for pharmaceutical active ingredients.

EP 0 275 285 B1 describes a method for preparing a stable superparamagnetic fluid in which dispersing and stabilizing is carried out by using ultrasound. Ultrasound treatment has the disadvantage of this energy input destroying thermally or mechanically unstable substances which may have been applied to the particles.

### SUMMARY OF THE INVENTION

It is the object of the present invention to provide a system for transporting active substances in a biological system, which does not absolutely require a modification of the magnetic particles and which makes it possible to incorporate these particles both into aqueous and oily suspensions and in microemulsions, oil-in-water emulsions, water-in-oil emulsions and also water-in-oil-in-water emulsions.

Accordingly, the present invention relates to a stabilizer-free system for transporting active substances in a biological system of one or more active substances and magnetic particles, which is characterized in that the particles are provided with active substances on at least part of their surface.

In the present invention, stabilizer-free means that the magnetic particles carry active substances or are enveloped by these without the addition of excipients such as emulsifiers or surface coatings, as are described in the prior art. A treatment of particles loaded with active substances is likewise not necessary and is preferably ruled out. In the simplest embodiment of the present invention, the system of the invention comprises a magnetic particle and an active substance.

According to the invention, at least a part of the surface of the magnetic particles is provided with active substance(s), meaning that the active substance molecules are applied directly to the particle surface. The particles may also be enveloped by the active substance, which is the case, for example, if the active substances, due to their structure, form or size, surround the magnetic particles, but are not directly attached to the surface. An envelope is present, for example, when the active substances used are cells, cell cultures or cell components and the magnetic particles are present inside the cells, cell cultures or cell components or when the active substances, due to their molecular size, form a globular structure inside which the magnetic particles are located.

The system of the invention may be used for the targeted transport of active substances to a specific site of action in the organism or the biological system and also for “taking away” unwanted components in/on the organism or from the organism. It has the advantage that the active substances are applied directly to the particles and thus the system of the invention comprises, in the simplest case, active substance(s) and magnetic particles.

It has been found that, on the one hand, the specific loading with active substance results in a kind of carrier function of the particles in the surrounding medium and, on the other hand, it is possible to selectively concentrate the loaded particles (sedimentation) and to concentrate the particles by applying a magnetic field. A relatively large particle surface area compared to larger particles makes it possible to load the surface with more active substances, i.e. the system of the invention can take up a distinctly higher concentration of active substances compared to the prior art. Overall it is possible to introduce the same active substance contents into the biological system with less magnetic material.

Biological system in accordance with the present invention means both a human or animal organism itself and an extracorporeal system such as, for example, cells/cell cultures eluted from the organism and/or extracorporeal apparatuses in which body fluids are purified. The particles of the invention are usually taken up by the organs, tissues and cells and also implants.

The magnetic particles used are in particular super-paramagnetic particles, in particular metal oxides or metals. Superparamagnetic particles have no remanence, i.e. they can be reversibly moved and concentrated in a magnetic gradient field.

The advantage of the magnetic particles used is in particular the fact that they are constructed entirely from inorganic material and can readily be sedimented in a magnetic field. In order to use the particles for transporting active substances in the biological system, no further components for the modification of the particles themselves, such as coating with polymers, etc., are required. The system can be suited to and set up for the particular application purpose in any manner.

Examples of suitable magnetic particles are γ-Fe₂O₃, Fe₃O₄, MnFe₂O₄, NiFe₂O₄, CoFe₂O₄ and any mixtures thereof, Fe₃O₄ (magnetite) being particularly preferably used. Possible metals which may be mentioned are Fe, Co, Ni and the alloys thereof, where appropriate also with other metals.

The magnetic particles used according to the invention preferably have a particle size of from 1 to 300 nm, preferably up to 100 nm, referring here to the individual discrete crystallites. Agglomerates whose overall particle size is above 100 nm, in particular above 300 nm, may also be present.

The volume-weighted average crystallite size can be determined using X-ray diffraction methods, in particular via a Scherrer analysis. The method is described, for example, in: C. E. Krill, R. Birringer: “Measuring average grain sizes in nanocrystalline materials”, Phil. Mag. A 77, p. 621, (1998). According to this, the volume-weighted average crystallite size D can be determined by the relation
D=Kλ/βcos θ.

λ is the wavelength of the X-radiation used, β is the full width at half the height of the reflection at diffraction position 2θ. K is a constant of the magnitude 1 whose exact value depends on the crystal form. This uncertainty of K can be avoided by determining the line broadening as integral width βᵢ, βᵢ being defined as the area under the X-ray diffraction reflection divided by the maximum intensity Iₒ of the latter:

$${β}_{i} = 1 / {I}_{0} {\int }_{2 {θ}_{1}}^{2 {θ}_{2}} I ( 2 θ ) ⅆ ( 2 θ )$$

The parameters 2θ₁ and 2θ₂ are the minimum and maximum angle positions of the Bragg reflection on the 2θ axis. I(2θ) is the measured intensity of the reflection as a function of 2θ. Using this relation gives the equation for determining the volume-weighted average crystallite size D: D=λ/βᵢcosθ.

In a possible embodiment of the present invention, the magnetic particles used are nanoparticles having a particle size of preferably less than 100 nm. The active substance used can be adsorbed to these particles, and it has proved particularly advantageous for the active substance to be present already at the formation of the magnetic particles, for example by size-controlled precipitation in aqueous medium by means of alkaline substances or by reduction of metal cations. The large particle surface area generated in situ makes possible an optimal absorption of the active substance to the surface by means of functional, preferably ionic or polar, groups in the active substance molecule, such as OH, SH, hydroxide, amino, carboxyl, ether, sulpho, phosphonic acid groups, etc. It is also possible to apply the active substance to the precipitated particles subsequently, for example by suspending the uncoated (non-modified) magnetic particles in a liquid phase containing the active substance or the active substance mixture, preferably water.

In a further possible embodiment of the present invention, the active substances may also be bound to the magnetic particles via spacer groups. Spacers are short organic molecule chains which are utilized when immobilizing molecules to supports; the spacer molecules do not constitute a coating. Spacers may be used, for example, if the active substances do not contain any polar groups or ionic groups. The spacer molecules can improve binding between magnetic particles and the active substances. They have preferably one or more polar groups. Examples which may be referred to are the previously mentioned groups. Spacers having two polar groups, such as aminocarboxylic acids, diamines, betaines, dicarboxylic acids, aminophosphonates, etc., have proved to be useful, in particular when using cationic active substances.

In another embodiment, “agglomerates” of magnetic particles are used, which consist of agglomerates of nanoparticles, i.e. of crystallites having a particle size of less than 100 nm. These agglomerates may be composed of individual crystallites which are either reversibly agglomerated at their contact surface area or irreversibly agglomerated by coalescence, i.e. by growing together across the grain boundaries. Agglomerates have the advantage of having both an outer and an inner surface, i.e. of having cavities, so that the active substances can be bound on the inside and on the outside. Agglomerates may be obtained, for example, by precipitating the magnetic particles in the absence of an active ingredient, by drying or freeze-drying active ingredient-free or active ingredient-loaded nanoparticles with subsequent redispersion, agglomerate formation which can be controlled via the synthesis conditions, for example increasing the temperature, adjusting the pH, high electrolyte content, or by a suitable aftertreatment of the precipitated particles at temperatures above 100° C.

Active substances in accordance with the present invention are both substances which are introduced into the organism and substances which are to be removed from it, for example synthetic pharmaceutical active ingredients, natural pharmaceutical active ingredients and extracts, natural and recombinant peptides, proteins, enzymes, antibodies and antibody fragments, endogenous biological units such as living and death cells, cell components and organelles, synthetic and natural DNA, genes, chromosomes, genetically modified autologous or heterologous cells and xenobiotic units such as bacteria, viruses, mycoplasma, fungi and spores, heat-conductive substances such as metals, radiologically active substances such as γ-radiators, particulate exogenous units containing active substances, such as liposomes, microcapsules and nanoparticles, and any mixtures of the above.

In a particularly preferred embodiment of this design, the active substances are selected from water-soluble and/or lipid-soluble pharmaceutical active ingredients.

In a preferred embodiment of the present invention, the active substances used are geminal bisphosphonic acids and/or the physiologically tolerated salts thereof, preferably those of the general formula I: 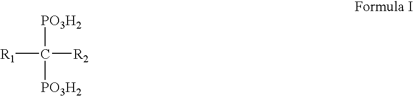
in which
R₁ is a linear or branched alkyl radical having from 1 to 10 carbon atoms, which is unsubstituted or substituted with substituents such as amino groups, N-mono- or N-dialkylamino groups, where the alkyl groups may contain from 1 to 5 carbon atoms and/or SH groups, or a substituted or unsubstituted carbocyclic or heterocyclic aryl radical which may have, where appropriate, one or more hetero atoms and, as substituents, branched and unbranched alkyl radicals having from 1 to 6 carbon atoms, free or mono- and, respectively, dialkylated amino groups having from 1 to 6 carbon atoms or halogen atoms, and
R₂ is OH, a halogen atom, preferably Cl, H or NH₂.

Examples of suitable salts of the compounds of the formula I, which may be mentioned, are alkali metal salts, ammonium salts and ethanolamine salts.

Such compounds are suitable in particular for the treatment of osteoporotic disorders, the following compounds being particularly preferred: 3-(methyl-pentylamino)-1-hydroxypropane 1,1-diphosphonic acid (ibandronic acid), 1-hydroxyethane 1,1-diphosphonic acid (etidronic acid), dichloromethanediphosphonic acid (clodronic acid), 3-amino-1-hydroxypropane 1,1-diphosphonic acid (pamidronic acid), 4-amino-1-hydroxybutane 1,1-diphosphonic acid (alendronic acid), 2-(3-pyridine)-1-hydroxyethane 1,1-diphosphonic acid (risedronic acid), 4-chlorophenylthiomethane 1,1-diphosphonic acid (tiludronic acid), pyrimidinyl-1-hydroxyethane 1,1-diphosphonic acid (zoledronic acid), cycloheptylaminomethane 1,1-diphosphonic acid (cimadronic acid), 6-amino-1-hydroxyhexane 1,1-diphosphonic acid (neridronic acid), 3-(N,N-dimethylamino)-1-hydroxypropane 1,1-diphosphonic acid (olpadronic acid), 3-pyrrole-1-hydroxypropane 1,1-diphosphonic acid and/or 2-pyrimidazole-1-hydroxyethane 1,1-diphosphonic acid (minodronic acid)

and the physiologically tolerated salts thereof.

As already mentioned above, the system of the invention comprises in its simplest form a magnetic particle or magnetic particles and one or more active substances. This system can be transferred in a manner known per se to a pharmaceutical preparation for oral, parenteral, intravenous, inhalative and/or topical administration and be administered to the biological system. Suitable forms of the pharmaceutical preparation which may be mentioned are suspensions, emulsions and liposomal systems.

In a possible embodiment, the system of the invention is a suspension. The preferred suspension medium is water or a physiological NaCl solution.

In a further aspect of the present invention, the inventive system for transporting active substances is an emulsion which can be a water-in-oil emulsion or an oil-in-water emulsion. The magnetic particles are preferably present exclusively in the oil phase which forms the inner phase, i.e. the droplets. In addition, magnetic particles may also be concentrated in the water phase. Those cases in which the magnetic particles are present in the oil phase are also referred to as oil-based ferrofluids. Both macroemulsions and microemulsions may be used, i.e. thermo-dynamically stable emulsion systems with droplet sizes of <500 nm.

Oil-based ferrofluids may be used as supports for lipid-soluble active substances so that both the magnetizable particles and the active substance(s) are present in the oil phase. In a further aspect, water-soluble active substances are used which are present in dissolved form in the aqueous phase, with the magnetic particles being in the oil phase.

In order to increase the physiological tolerability and to effect a rapid distribution of the active substances in the bloodstream, the loaded particles may be emulsified in water, where appropriate also by using suitable physiologically tolerated emulsifiers. An example of a commercial emulsifier is Solutol® (BASF AG). If the active substance and the magnetic particles are present in the oil phase, the active ingredients may be bound (absorbed) to the magnetic particles here, too, but this is not absolutely necessary.

In a further aspect, water-soluble active ingredients may be dissolved or suspended in the water phase of an oil-based ferrofluid emulsion. This embodiment is particularly useful if the active substances are water-soluble polymers such as cell components, proteins, etc. With the aid of suitable emulsifiers, it is possible to transfer a water phase containing active substances of this kind into an emulsion containing magnetic oil. The emulsion mixture can then be concentrated at the site of action by means of a permanent magnetic field.

In a preferred embodiment, the active substances and magnetic particles which are present independently of one another in the aqueous phase are enclosed in the aqueous interior space of liposomes. If desired, the fraction which is not enclosed can be removed by centrifugation or gel chromatography, resulting in a purified magnetic liposome product. The same process can be applied in order to encapsulate liposomally the above-described magnetic particles with adsorbed active substances.

In a possible embodiment of the present invention, the active substances are adsorbed to the magnetic particles and are, at the same time, present in free form in the aqueous phase and/or in the oil phase. The adsorption is carried out either during formation of the magnetic particles via precipitation or by suspending the magnetic particles in a solution, a suspension or a dispersion of the active substances. The loaded particles may be formulated in each case in the form of a suspension, microemulsion, oil-in-water emulsion, water-in-oil emulsion, water-in-oil-in-water emulsion, etc. The loaded magnetic particles may be concentrated in the aqueous phase or in the oil phase.

The inventive system for transporting active substances in a biological system can be prepared in various ways.

In a first embodiment, a water-soluble or in water suspendable active substance and a water-soluble precursor of the magnetic particles are dissolved in water and the magnetic particles are formed by precipitation, with the magnetic particles precipitating as solids loaded with the active substance.

In a further aspect of the present preparation method, the magnetic particles are added to a solution or suspension of the active substance in water or another liquid. The magnetic particles are loaded with active substance by adsorption to the surface of the magnetic particles.

In a further aspect, oil-based ferrofluids may be obtained. Preferably, the magnetic particles are first admixed with a solid or liquid oil or molten wax with stirring and, where appropriate, with heating. The lipid-soluble particles obtained can then be emulsified in water in the presence of the active substance in a manner known per se.

Oils or waxes which may be used are all natural or synthetic oils or waxes which are suitable for the particular field of use and are liquid at the processing temperature, as long as they are pharmaceutically acceptable. If the oils or waxes are in solid form, they may be heated in order to prepare an oil-based ferrofluid.

In a further aspect of the preparation method, lipid-soluble active substances may be used in the system of the invention. In a preferred embodiment, these active substances are first admixed with the lipid-soluble particles obtained and the mixture is then emulsified in water. In a further possible aspect, the lipid-soluble active substances can be added to the magnetic particles, before or while the latter are admixed with the solid or liquid oil or wax. In a preferred aspect, these active substances are first admixed with the lipid-soluble particles obtained and the mixture is then emulsified in water.

The present invention further relates to the use of the above-described system for transporting active substances in a biological system for the targeted transport of pharmaceutically active ingredients in the biological system and to the use of the system for concentrating active substances in the biological system at predetermined locations. For this purpose, the ferrofluid medicinal product can be converted or incorporated into suitable conventional pharmaceutical forms. For example, a pharmaceutical form suitable for oral administration of a medicament-loaded oil-based ferrofluid is a soft gelatin capsule. A form particularly suitable for systemic injection or inhalative application is the liposomal encapsulation as described above. Aqueous suspensions or oil-in-water emulsions are particularly suitable for introduction and positioning in body cavities such as the peritoneum, bladder space, urogenital tract and vaginal tract. The active substances used can be transported specifically, for example, by applying externally a magnetic field at or close to the location to be treated, thereby concentrating the active substance locally at the desired site (drug targeting).

At the target organ or target location, the active substances can exert their action according to their activity and, where appropriate, be released from the particles.

Active substances which are intended to exert their action directly by contacting the biological system to be treated are preferably released directly at the site of action. An example of active substances of this kind is the action of chemotherapeutics, cytostatics, therapy-supporting active ingredients such as anti-inflammatory agents, painkillers, etc., which are transported to their site of action via transport by the magnetic particles by means of an applied magnetic field, released there, owing to their affinity for the tissue, tumour, or the like to be treated and then exert their action. After the release is completed, the magnetic particles can be removed again from the organism via the applied magnetic field, that means after altering the position of the magnetic field.

If the active substances used are, for example, the above-described bisphosphonates, it is possible, for the treatment of bone tumours or metastases in the bones, to transport the system of the invention, a ferrofluid of magnetic particles and bisphosphonate, to the tumour. The bisphosphonate binds to the bone apatite and inhibits bone conversion. The external magnetic field locally heats and thus destroys the tumour cells.

In a further embodiment, active substances which display radiological properties, such as γ-radiators, etc., can be transported according to the invention to their site of action and there destroy the tissue to be treated by local irradiation, where appropriate under external heating. After irradiation, the particles can be removed again by means of a magnetic field.

Besides the targeting effect, the use of the magnetic particles has the additional advantage that the permanent magnetization of the particles promotes the release of the active substances. The permanent magnetization can result in a local overheating which supports the release of active substances which are only loosely applied to the particle surface. Another release mechanism which occurs in particular if there is no permanent magnetization is the slow dissociation of the active substance from the magnetic particle. This release may take place via chemical, in particular enzymatic, hydrolytic or thermal removal or else via a purely physical removal. Thermal removal of the active substances from the magnetic particles is supported preferably by an applied magnetic field.

Another advantage is the local overheating which occurs with permanent magnetization of the magnetic particles present at the local site of action. This overheating may be utilized in order to destroy diseased tissue or tumour tissue. Local overheating by means of using a magnetic field is also referred to as local or cellular hyperthermia.

The combination of the system of the invention and hyperthermia can also be used, for example, in the treatment of tumours which can be treated using “thermoseeds”. The thermoseeds normally consist of an alloy of a magnetic metal such as iron or cobalt and a non-magnetic material such as the noble metals gold, silver, palladium or platinum. After implanting the thermoseeds into the tumour, the system of the invention, which contains, for example, magnetic particles and a chemotherapeutic as active substance, can be administered. The system of the invention accumulates in the thermoseeds and thus forms a local depot of cytostatics. Following the accumulation of the system of the invention at the tumour, the usual thermoseed treatment can be carried out by emitting extracorporeally an alternating magnetic field which causes heating of the thermoseeds and, connected therewith, destruction of the tumour cells.

A similar, tumour-treating action can be achieved if the active substance used is a metal with good heat conductivity, such as palladium or platinum. In this embodiment, the inventive system of magnetic particle and metal is transported to its site of action, followed by heating to the Curie point by applying externally an alternating magnetic field. The ensuing limited overheating causes destruction of the tumour cells. This embodiment may be applied to any tumour. The system of the invention may be injected within a suitable pharmaceutical preparation, as described above, or else inhaled. For example, inhalation of this system is suitable for the treatment of lung tumours.

### PREFERRED EMBODIMENTS

### Example 1

### Preparation of a Water-Dispersed Ferrofluid

6.48 g of FeCl₃ were dissolved in 40 g of deionized water. In addition, 3.97 g of FeCl₂.4H₂O were dissolved in a mixture of 8 ml of deionized water and 2 ml of 37% strength hydrochloric acid. The two mixtures were combined shortly before using the solutions in the precipitation process.

400 ml deionized water were mixed with stirring with 10 g of NaOH and 0.2 g of hydroxyethanediphosphonic acid (HEDP) in a glass beaker. After cooling, the hydrochloric iron salt solution was added to this with vigorous stirring. The black precipitate formed was sedimented by means of a magnetic field and the supernatant was decanted off. The precipitated material was then taken up in water and decanted several times, in order to remove extraneous ions. Then 0.5 g of HEDP and 100 ml of water were added. After stirring at 40° C. for 1 hour, stirring was continued at room temperature for 12 h. Unsuspended portions were removed by centrifugation (5,000-11,000 revolutions/minute). In this way a magnetic fluid was obtained which was concentrated in a rotary evaporator until the desired solid content was obtained.

### Example 2

### Preparation of an Oil-Based Ferrofluid-in-Water Emulsion

a. Preparation of the Oil-Based Ferrofluid

7.8 g of anhydrous ferric chloride were dissolved in 50 g of CO₂-free water. At the same time, 4.8 g of FeCl₂.4H₂O were dissolved in 10 g of water in a second vessel and acidified with hydrochloric acid to a pH of 2. Both solutions were then combined and added to a vigorously stirred solution in another vessel, which consists of 100 ml of 25% strength ammonia solution and 300 ml of deionized water, with precipitation of a black precipitate. After washing several times with water and removing in each case the supernatant aqueous phase by centrifugation and decanting, the precipitate was admixed with 100 g of water and 2.0 g of lauric acid. The mixture was heated with stirring to 85° C. until the precipitate sedimented with the formation of flocks. Subsequently, 10 g of sunflower oil were added to the mixture which was still at 85° C. and was then stirred for one hour. During this process, the precipitate was taken up in a dispersion-stable manner in the oil phase which was removed and extracted several times with water. An oil-based ferrofluid was obtained.

b. Transport of an Active Substance to a Diseased Joint

(Active Substance: Antirheumatic Agent Nabumetone)

The oil ferrofluid obtained was admixed with isotonic saline in a volume ratio of 1:9. A ferrofluid-in-water emulsion with droplet sizes in the range of 10-100 μm was obtained by adding the emulsifier Solutol® HS 15 (polyethylene glycol 660-12-hydroxystearate, manufacturer: BASF AG) with a proportion by weight of 15 g per litre and subsequent stirring by means of a magnetic stirrer. Loading with the oil-soluble active ingredient (nabumetone) was carried out prior to emulsification by dissolving 2 ml of active ingredient in 8 ml of oil-based ferrofluid.

### Example 3

### Preparation of a Ferrofluid in which the Active Substance Corresponds to the Oil Phase

7.8 g of anhydrous ferric chloride were dissolved in 50 g of CO₂-free water. In a second vessel, 4.8 g of FeCl₂.4H₂O were dissolved in 10 g of water and the solution obtained was acidified with hydrochloric acid to a pH of 2. Both solutions were combined to a mixture and added to a vigorously stirred solution in another vessel, consisting of 100 ml of 25% ammonia solution and 300 ml of deionized water, with precipitation of a black precipitate. After washing several times with water and removing in each case the supernatant aqueous phase by centrifugation and decanting, the precipitate was admixed with 100 g of water and 2.0 g of lauric acid. The mixture was heated with stirring to 85° C. until the precipitate sedimented with the formation of flocks. Subsequently, 10 g of the oil-soluble active ingredient nabumetone were added to the mixture which was still at 85° C. and was then stirred for one hour. During this process, the precipitate was taken up in a dispersion-stable manner in the oil phase which was removed and extracted several times with water. An oil-based ferrofluid was obtained.

The oil-based ferrofluid was admixed with isotonic saline in a volume ratio of 1:9. A ferrofluid-in-water emulsion with droplet sizes in the range of 5-100 micrometer was obtained by adding the emulsifier Solutol® HS 15 (polyethylene glycol 660-12-hydroxystearate, manufacturer: BASF AG) with a proportion by weight of 15 g per litre and subsequent stirring by means of a magnetic stirrer. For an active ingredient dosage, the oil phase was diluted appropriately with isotonic saline.

### Example 4

### Liposomally Encapsulated Water-Dispersed Ferrofluid

5 g of Phospholipone are dissolved in chloroform in a round-bottomed flask and the organic phase is stripped off in a rotary evaporator under reduced pressure until a thin, solvent-free lipid film has formed. 100 ml of the water-dispersed ferrofluid product of Example 1 are added to the lipid film with gentle heating and agitated on a mechanical shaker for one hour, until the lipid film has detached completely from the wall and liposomes have formed. The preparation is treated with ultrasound for 1-2 minutes in order to achieve a liposome size distribution in the nanometer range. The preparation is then separated from the non-encapsulated fraction via a Sephadex-G75 column. The ferrofluid-liposome dispersion can be adjusted to the desired concentration with isotonic saline.

### Example 5

### Aqueous Solutions of Natural or Synthetic Proteins

1 ml of an aqueous solution of urodilatin (nephroprotective protein) was emulsified in 9 ml of the oil-based ferrofluid of Example 2 at room temperature by adding 1 g of Solutol® HS 15 and stirring by means of a magnetic stirrer. A water-in-ferrofluid emulsion was obtained, which was emulsified in 30 ml of a 1% strength aqueous Solutol solution by means of magnetic stirring. A protein-containing water-in-ferrofluid-in-water emulsion with droplet sizes in the range of 3-50 micrometer formed.

The ferrofluid is injected and transports the magnet-supported urodilatin specifically to the site of action, namely the kidneys. This is followed by desorption and separation of the unloaded particles from the blood via external haemodialysis, i.e. ferroparticles are captured and removed by magnetic blood adsorbers.

### Example 6

### Magnetic Transport of DNA in a Water-in-Oil-in-Water Emulsion

1 ml of an isotonic salt solution of the oligonucleotide single-strand 24mer phosphorothioate (synthetic DNA derivative) was emulsified in 9 ml of the oil-based ferrofluid of Example 2 by addition of 1 g of Solutol® HS 15 and vigorous stirring by means of a magnetic stirrer at room temperature. A water-in-ferrofluid emulsion was obtained which had no long-term stability and was emulsified in 30 ml of a 1% strength aqueous Solutol solution by means of magnetic stirring. An oligonucleotide-containing water-in-ferrofluid-in-water emulsion with droplet sizes in the micrometer range formed.

### Example 7

### Magnetic Transport of Cells and Cell Components in a Water-in-Oil-in-Water Emulsion

1 ml of an isotonic salt solution of genetically modified epithelial cells was emulsified in 9 ml of ferrofluid obtained in Example 2 by addition of 1 g of Solutol® HS 15 and stirring by means of a magnetic stirrer at room temperature. A water-in-ferrofluid emulsion which had no long-term stability was obtained and emulsified in 30 ml of a 1% strength aqueous Solutol solution by means of magnetic stirring. A cell-containing water-in-ferrofluid-in-water emulsion with droplet sizes in the micrometer range formed.

The cell-containing ferrofluid emulsion obtained is suitable, for example, for targeted cell transport to and placing at a specific site of action, for example for attaching the cells to particular sites on vascular walls (e.g. in coronary arteries after PTCA).

What is claimed is:

1. A system for transporting active substances in a biological system; the system consisting of:
magnetic particles having a surface without a surface coating;
one or more active substances directly applied to at least part of the surface of the magnetic particles without any stabilizer being present in the system.

2. System according to claim 1, wherein the biological system is a human or animal organism, an extracorporeal system such as cells/cell cultures eluted from the organism and/or extracorporeal apparatuses in which body fluids are purified.

3. System according to claim 1, wherein the magnetic particles are selected from γ-Fe₂O₃, Fe₃O₄, MnFe₂O₄, CoFe₂O₄ and any mixtures thereof.

4. System according to claim 1, wherein the magnetic particles have a particle size of from 1 to 300 nm, preferably up to 100 nm.

5. System according to claim 1, wherein the active substances are selected from substances which are introduced into the organism and from substances which are to be removed from it.

6. System according to claim 1, wherein the active substances are selected from synthetic pharmaceutical active ingredients, natural pharmaceutical active ingredients and extracts, natural and recombinant peptides, proteins, enzymes, antibodies and antibody fragments, endogenous biological units such as living and death cells, cell components and organelles, synthetic and natural DNA, genes, chromosomes, genetically modified autologous or heterologous cells and xenobiotic units such as bacteria, viruses, mycoplasma, fungi and spores, heat-conductive substances such as metals, radiologically active substances such as γ-radiators, particulate exogenous units containing active substances, such as liposomes, microcapsules and nanoparticles, and any mixtures of the above.

7. System according to claim 1, wherein the active substances are selected from water-soluble active substances are selected from geminal bisphosphonic acids and/or the physiologically tolerated salts thereof of the general formula I 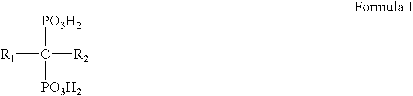
in which
R₁ is a linear or branched alkyl radical having from 1 to 10 carbon atoms, which is unsubstituted or substituted with substituents such as amino groups, N-mono- or N-dialkylamino groups, where the alkyl groups may contain from 1 to 5 carbon atoms and/or SH groups, or a substituted or unsubstituted carbocyclic or heterocyclic aryl radical which may have, where appropriate, one or more hetero atoms and, as substituents, branched and unbranched alkyl radicals having from 1 to 6 carbon atoms, free or mono- and, respectively, dialkylated amino groups having from 1 to 6 carbon atoms or halogen atoms, and
R₂ is OH, a halogen atom, preferably Cl, H or NH₂.

8. System according to claim 1, wherein the bisphosphonate is selected from 3-(methyl-pentylamino)-1-hydroxypropane 1,1-diphosphonic acid, 1-hydroxyethane 1,1-diphosphonic acid, dichloromethanediphosphonic acid, 3-amino-1-hydroxypropane 1,1-diphosphonic acid, 4-amino-1-hydroxybutane 1,1-diphosphonic acid, 2-(3-pyridine)-1-hydroxyethane 1,1-diphosphonic acid, 4-chlorophenylthiomethane 1,1-diphosphonic acid, pyrimidinyl-1-hydroxyethane 1,1-diphosphonic acid, cycloheptylaminomethane 1,1-diphosphonic acid, 6-amino-1-hydroxyhexane 1,1-diphosphonic acid, 3-(N,N-dimethylamino)-1-hydroxypropane 1,1-diphosphonic acid, 3-pyrrole-1-hydroxypropane 1,1-diphosphonic acid and/or 2-pyrimidazole-1-hydroxyethane 1,1-diphosphonic acid and the physiologically tolerated salts thereof.

9. System according to claim 1, wherein it is a suspension, emulsion, or liposomal system and it is transferred to a pharmaceutical preparation for oral, parenteral, intravenous, inhalative and/or topical administration.

10. Method for preparing a system for transporting active substances according to claim 1, wherein a water-soluble or water-suspendable active substance and a water-soluble precursor of the magnetic particles are dissolved in water and the magnetic particles are formed by precipitation, the magnetic particles precipitating as solids loaded with the active substance.

11. Method for preparing a system for transporting active substances according to claim 1, wherein the magnetic particles are introduced into a solution or suspension of the active substance in water or another liquid.

12. A system for transporting active substances in a biological system; the system consisting of:
magnetic particles having a surface without a surface coating;
one or more active substances, wherein the magnetic particles carry the one or more active substances or are enveloped by the one or more active substances without any stabilizer being present in the system.

13. System according to claim 12, wherein the biological system is a human or animal organism, an extracorporeal system such as cells/cell cultures eluted from the organism and/or extracorporeal apparatuses in which body fluids are purified.

14. System according to claim 12, wherein the magnetic particles are selected from γ-Fe₂O₃, Fe₃O₄, MnFe₂O₄, CoFe₂O₄ and any mixtures thereof.

15. System according to claim 12, wherein the magnetic particles have a particle size of from 1 to 300 nm, preferably up to 100 nm.

16. System according to claim 12, wherein the active substances are selected from substances which are introduced into the organism and from substances which are to be removed from it.

17. System according to claim 12, wherein the active substances are selected from synthetic pharmaceutical active ingredients, natural pharmaceutical active ingredients and extracts, natural and recombinant peptides, proteins, enzymes, antibodies and antibody fragments, endogenous biological units such as living and death cells, cell components and organelles, synthetic and natural DNA, genes, chromosomes, genetically modified autologous or heterologous cells and xenobiotic units such as bacteria, viruses, mycoplasma, fungi and spores, heat-conductive substances such as metals, radiologically active substances such as γ-radiators, particulate exogenous units containing active substances, such as liposomes, microcapsules and nanoparticles, and any mixtures of the above.

18. System according to claim 12, wherein the active substances are selected from water-soluble active substances are selected from geminal bisphosphonic acids and/or the physiologically tolerated salts thereof of the general formula I 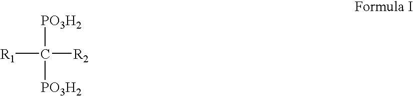
in which
R₁ is a linear or branched alkyl radical having from 1 to 10 carbon atoms, which is unsubstituted or substituted with substituents such as amino groups, N-mono- or N-dialkylamino groups, where the alkyl groups may contain from 1 to 5 carbon atoms and/or SH groups, or a substituted or unsubstituted carbocyclic or heterocyclic aryl radical which may have, where appropriate, one or more hetero atoms and, as substituents, branched and unbranched alkyl radicals having from 1 to 6 carbon atoms, free or mono- and, respectively, dialkylated amino groups having from 1 to 6 carbon atoms or halogen atoms, and
R₂ is OH, a halogen atom, preferably Cl, H or NH₂.

19. System according to claim 12, wherein the bisphosphonate is selected from 3-(methyl-pentylamino)-1-hydroxypropane 1,1-diphosphonic acid, 1-hydroxyethane 1,1-diphosphonic acid, dichloromethanediphosphonic acid, 3-amino-1-hydroxypropane 1,1-diphosphonic acid, 4-amino-1-hydroxybutane 1,1-diphosphonic acid, 2-(3-pyridine)-1-hydroxyethane 1,1-diphosphonic acid, 4-chlorophenylthiomethane 1,1-diphosphonic acid, pyrimidinyl-1-hydroxyethane 1,1-diphosphonic acid, cycloheptylaminomethane 1,1-diphosphonic acid, 6-amino-1-hydroxyhexane 1,1-diphosphonic acid, 3-(N,N-dimethylamino)-1-hydroxypropane 1,1-diphosphonic acid, 3-pyrrole-1-hydroxypropane 1,1-diphosphonic acid and/or 2-pyrimidazole-1-hydroxyethane 1,1-diphosphonic acid and the physiologically tolerated salts thereof.

20. System according to claim 12, wherein it is a suspension, emulsion, or liposomal system and it is transferred to a pharmaceutical preparation for oral, parenteral, intravenous, inhalative and/or topical administration.

21. Method for preparing a system for transporting active substances according to claim 12, wherein a water-soluble or water-suspendable active substance and a water-soluble precursor of the magnetic particles are dissolved in water and the magnetic particles are formed by precipitation, the magnetic particles precipitating as solids loaded with the active substance.

22. Method for preparing a system for transporting active substances according to claim 12, wherein the magnetic particles are introduced into a solution or suspension of the active substance in water or another liquid.
